(19) United States
(12) Patent Application Publication
(10) Pub. No.: US 2026/0083308 A1
(43) Pub. Date: Mar. 26, 2026

(54) GASTROESOPHAGEAL TAMPONADE AND METHOD OF USE

(71) Applicant: The Regents of the University of Michigan, Ann Arbor, MI (US)

(72) Inventors: Amanda CHEUNG, Ann Arbor, MI (US); Natalie TRAKANRUNGSI, Ann Arbor, MI (US); David KAUFMAN, Ann Arbor, MI (US); Ella KOPPLIN, Hailey, ID (US); Neil BHATE, Ann Arbor, MI (US)

(21) Appl. No.: 19/333,928

(22) Filed: Sep. 19, 2025

Related U.S. Application Data
(60) Provisional application No. 63/697,125, filed on Sep. 20, 2024.

(51) Int. Cl. A61B 1/06; A61B 1/273; A61B 17/12; A61B 90/92; A61B 17/00

(52) U.S. Cl. CPC A61B 1/0684; A61B 1/2733; A61B 17/1204; A61B 17/12099; A61B 17/12136; A61B 90/92; A61B 2017/00818; A61B 2017/12004; A61B 2217/005

(57) ABSTRACT

A gastroesophageal tamponade and method of using a gastroesophageal tamponade involves illuminating a trachea and an esophagus of a subject with light from a distal end of the tamponade. In one implementation, the gastroesophageal tamponade comprises a body extending from a proximal end to a distal end. The body has an outer perimeter and a guide lumen extending at least partially through the body. The body comprises an aspiration lumen located at least partially between the guide lumen and the outer perimeter and an inflation lumen located at least partially between the guide lumen and the outer perimeter. A balloon extends at least partially around the body.

### FIELD

This disclosure relates generally to tamponades, and more particularly, to gastroesophageal tamponades that are configured with one or more inflatable balloons.

### BACKGROUND

The process of inserting current esophageal balloon tamponade devices to stop esophageal bleeding is time-consuming, difficult, prone to user error, and dangerous. Approximately 40% of patients who receive an esophageal balloon tamponade as treatment do not survive to discharge due to currently available devices not being fully inserted before fatal bleeding occurs.

Esophageal bleeding occurs in cirrhosis patients who have ruptured esophageal varices. Cirrhosis is the final stage of many liver diseases and is characterized by irreversible fibrosis and failure of the liver, leading to an increase in morbidity and mortality. Cirrhosis affects roughly 0.3% of the U.S. population, which is around 600000 individuals.

The portal venous system drains blood from the gi-tract, spleen, pancreas, and gallbladder to the liver. Whenever the liver is injured, it tries to repair itself, thus creating scar tissue. When the scarring becomes excessive, the patient is diagnosed with cirrhosis. cirrhosis can be caused by excessive alcohol consumption, infections, and fatty liver caused by obesity or diabetes. Scar tissue buildup in the liver inhibits blood flow in the compressed portal vein, causing a backup in esophageal veins. cirrhosis leads to portal hypertension. This causes esophageal varices to form, increasing the risk of rupture of venous walls. Due to this, esophageal varices develop in 50% of cirrhosis patients, and 10-15% of these patients experience bleeding each year, accounting for up to 30,000 - 45,000 bleeding episodes. In 20% of bleeds, transfusions, vasoactive medications, and early endoscopy are unable to control the bleeding, contributing to a 10-20% mortality rate. Bleeding can also occur at the gastroesophageal junction, where the esophagus and stomach meet.

In these situations, gastroesophageal balloon tamponades are used by emergency physicians to temporarily halt esophageal bleeding before a GI surgeon will operate on the patient. With these tamponades, the gastric balloon is the most distal balloon and is placed in the patient’s stomach. When inflated, the gastric balloon keeps the device in place and stops bleeding at the gastroesophageal junction by applying pressure to the region. The esophageal balloon is the longer of the two balloons and is placed in the patient’s stomach; when inflated, it applies pressure to the esophageal region to stop bleeding. The gastric and esophageal inflation ports inflate the gastric and esophageal balloons, and the gastric and esophageal suction ports allow for suction of the gastric and esophageal regions, respectively. Two additional ports are present on both the gastric and esophageal inflation ports and are designed to allow for balloon pressure monitoring to ensure that the balloons retain the correct amount of pressure; however, according to physicians, these are rarely used.

Moreover, given that physicians are only trained on the procedure use of the tamponade in residency, many do not know how to insert the device, do not know they need to use stopcocks, do not know the components required, and more. Physicians struggle with insertion and are prone to mistakes due to lack of familiarity with the device. This unfamiliarity can be exacerbated in small, rural hospitals. In these areas, there is likely to be only one physician at the hospital who could perform the procedure, and any nurses present would not have experience with the device. Accordingly, improvements can be made to streamline the whole process so that a physician and nurse team with little experience inserting the device would be able to perform the procedure. Overall, physicians struggle with insertion and are prone to mistakes due to lack of familiarity with the device. Some hospitals do not even purchase the tamponades due to their difficult use.

There are similar sentiments that the insertion process of the tamponade is time-consuming, unintuitive, and prone to error. Critical design oversights such as compressible structure, ambiguous ports, and poor visual cues significantly increase the insertion time, which is not affordable when a patient is experiencing esophageal bleeding. The process becomes increasingly difficult in a rural ER due to the infrequency of the procedure, leading to very few physicians with experience performing the procedure, if any at all. The pitfalls associated with this device arise from, but are not limited to, lack of regular training, a convoluted procedure with many steps, and design decisions. Accordingly, there is a need for a solution that expedites the successful insertion of a device that temporarily stops esophageal bleeding in cirrhosis patients in order to provide necessary life support before surgical intervention.

Current devices have numerous shortcomings. Assembly is very time consuming, as prior to using the device, clinicians must gather syringes and 3-way stopcocks, ‘Christmas tree’ luer-lock converters for aspiration, a manometer to measure balloon inflation pressure, laryngoscopes, and lubrication, which can be scattered in various locations in the hospital. The device must also be placed in ice-water, as the device lacks stiffness and can coil during insertion. This adds yet another step to assembly. Device operation is another concern, as the ports of current devices are uniform in color and shape, and have inadequate labeling, which can make it difficult to distinguish the various ports. This also can add time to the process. Furthermore, relying on external measurement markings to determine insertion depth can be error-prone, as patient anatomy varies. Because of this, an X-ray is required to confirm that the gastric balloon is fully in the stomach. Following successful insertion, the tube is secured via a counterweight, created by tying the end of the tube to a one-pound bag of IV fluids and hanging it over an IV pole or by tying the end of the tube to a football helmet on the patient’s head. This can maintain the proper positioning of the gastric and esophageal balloon.

Based on these limitations, a potential solution should be stiffer and allow for easier insertion and have simpler ports and/or port labeling. The solution also should come in better packaging with all of the necessary components stored together and a proper external anchor. An ideal solution should also eliminate the need for radiological imaging and have a way to measure balloon pressure if application of pressure is the method used to stop bleeding.

### SUMMARY

In accordance with one embodiment, there is provided a gastroesophageal tamponade comprising a body extending from a proximal end to a distal end. The body has an outer perimeter and a guide lumen extending at least partially through the body. The body comprises an aspiration lumen located at least partially between the guide lumen and the outer perimeter and an inflation lumen located at least partially between the guide lumen and the outer perimeter. A balloon extends at least partially around the body.

In some embodiments, there is a guide wire located at least partially within the guide lumen. The guide wire can have a light source configured to illuminate an esophagus of a subject, with the light source being at a distal end of the guide wire. In some implementations, the light source is a white light emitting diode (LED). The distal end of the body can include a transparent or translucent sheath.

In some embodiments, the inflation lumen is an esophageal inflation lumen and the balloon is an esophageal balloon. The tamponade may also include a gastric inflation lumen and a gastric balloon. The gastric inflation lumen and the esophageal inflation lumen can be diametrically opposed with the guide lumen being a centrally located guide lumen. The aspiration lumen can be a gastric aspiration lumen, and the tamponade may also include an esophageal aspiration lumen. In some implementations, the gastric inflation lumen and the esophageal inflation lumen are diametrically opposed, and the gastric aspiration lumen and the esophageal aspiration lumen are diametrically opposed. A gastric inflation port can be coupled to the gastric inflation lumen, an esophageal inflation port can be coupled to the esophageal inflation lumen, a gastric aspiration port can be coupled to the gastric aspiration lumen, and an esophageal aspiration port can be coupled to the esophageal aspiration lumen. The gastric inflation port and the gastric aspiration port can be colored with one or more warm colors and the esophageal inflation port and the esophageal aspiration port can be colored with one or more cool colors. The gastric inflation port, the esophageal inflation port, the gastric aspiration port, and the esophageal aspiration port may be configured to extend radially outward from the body adjacent the guide lumen with the gastric inflation port being larger than the esophageal inflation port.

In some embodiments, the aspiration lumen has a cross-sectional shape and the inflation lumen has a cross-sectional shape, with the cross-sectional shape of the aspiration lumen being different from the cross-sectional shape of the inflation lumen. A cross-sectional area of the cross-sectional shape of the aspiration lumen can be larger than a cross-sectional area of the cross-sectional shape of the inflation lumen.

In accordance with another embodiment, there is provided a gastroesophageal tamponade comprising a body extending from a proximal end to a distal end, with a guide lumen extending at least partially through the body. The tamponade includes a balloon extending at least partially around the body, a guide wire located at least partially within the guide lumen of the body, and a light source configured to illuminate an esophagus of a patient. The guide lumen can be a centrally located guide lumen, with the body including a plurality of aspiration lumens and a plurality of inflation lumens. The plurality of aspiration lumens and the plurality of inflation lumens surround the centrally located guide lumen.

In accordance with another embodiment, there is provided a method of using a gastroesophageal tamponade, the gastroesophageal tamponade comprising a body extending from a proximal end to a distal end and a light source configured to illuminate the distal end of the body. The method comprises the steps of illuminating a trachea and an esophagus of a subject with light from the light source, and inserting the body of the gastroesophageal tamponade into the esophagus of the subject.

### BRIEF DESCRIPTION OF THE DRAWINGS

Preferred example embodiments will hereinafter be described in conjunction with the appended drawings, wherein like designations denote like elements, and wherein:

FIG. 1 shows a gastroesophageal tamponade in accordance with one embodiment;

FIG. 2 shows the gastroesophageal tamponade of FIG. 1;

FIG. 3 is a view of the proximal end of the gastroesophageal tamponade of FIGS. 1 and 2;

FIG. 4 is another view of the proximal end of the gastroesophageal tamponade of FIGS. 1-3;

FIG. 5 is a cross-section view of the body of the gastroesophageal tamponade of FIGS. 1-4;

FIG. 6 shows an example guide wire lumen end that could be used with the tamponade of FIGS. 1-4;

FIG. 7 shows example esophageal aspiration ends that could be used with the tamponade of FIGS. 1-4;

FIG. 8 shows example gastric aspiration ends that could be used with the tamponade of FIGS. 1-4;

FIG. 9 shows example esophageal inflation ends that could be used with the tamponade of FIGS. 1-4;

FIG. 10 shows another example guide wire lumen end that would be used with the tamponade of FIGS. 1-4;

FIG. 11 shows one embodiment of a distal end of a guide wire that can be used with the tamponade of FIGS. 1-4;

FIG. 12 shows another embodiment of a gastroesophageal tamponade;

FIG. 13 is a cross-section view of a body of a gastroesophageal tamponade in accordance with another embodiment; and

FIG. 14 is a cross-section view of a gastroesophageal tamponade in accordance with yet another embodiment.

Described herein is a gastroesophageal tamponade that is significantly quicker and more intuitive for physicians. Insertion is streamlined, as a guide wire is employed that helps avoid catheter impaction (bunching, folding) while progressing down the esophagus. Further structural details can be included which help avoid damage to the esophagus. Esophageal perforation and damage can occur if the force exerted on the esophageal wall when inserting the device exceeds 300 kPa, so the pressure exerted should not exceed this value. Additionally, the device must accommodate the average esophagus size, which can be a challenge when adding lumens and guide wire functionality, as detailed herein.

The device is designed to be deployed within the gastrointestinal tract, and users must distinguish between the trachea and esophagus to ensure proper placement and avoid potential complications. Users' ability to differentiate between anatomical structures contributes to the overall user-friendliness of the device. Accordingly, a light source is included to help users visualize the oral cavity. The ports are also particularly designed to enhance differentiation, thereby making the device easier to use in emergency situations.

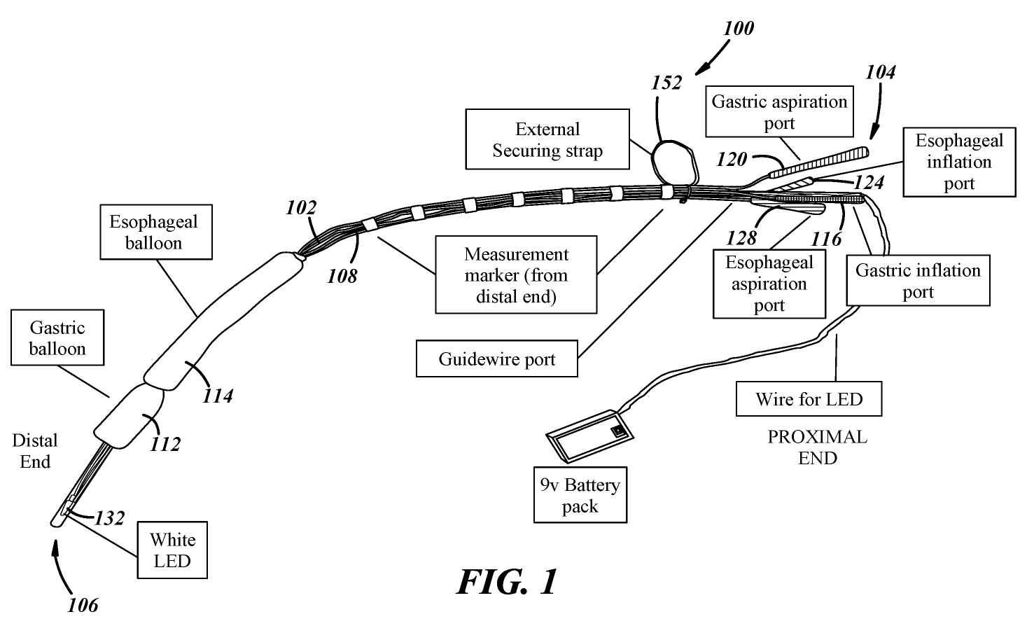

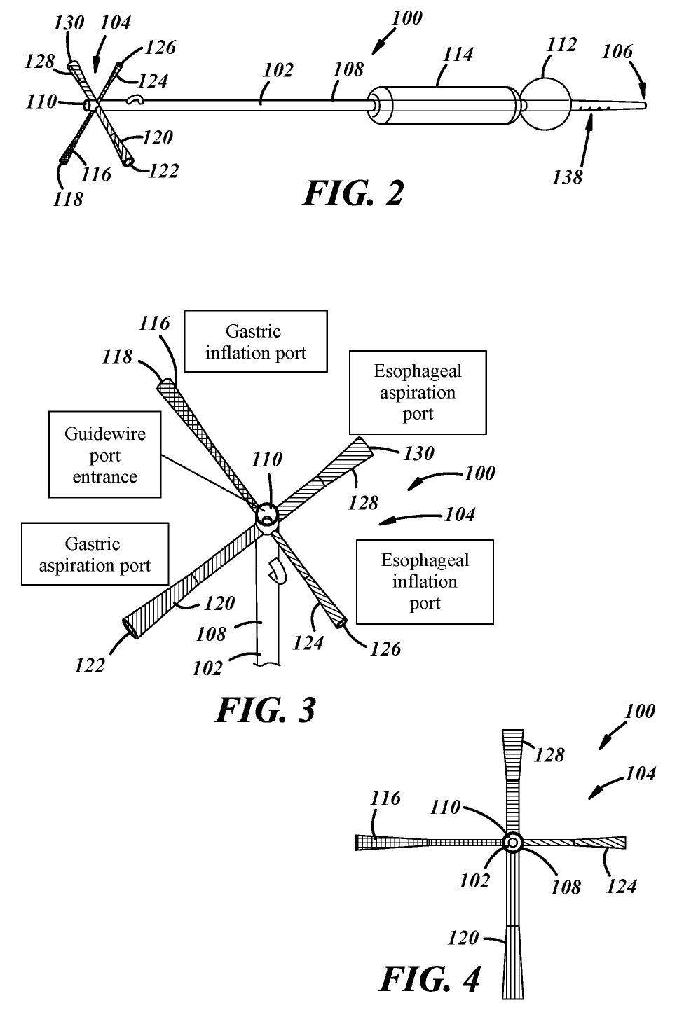

FIGS. 1-4 show an example gastroesophageal tamponade 100. The tamponade includes a body 102 extending from a proximal end 104 to a distal end 106. The body 102 has an outer perimeter 108 and a guide lumen 110 that extends at least partially through the body (see e.g., FIGS. 2-4), or in some implementations, fully through an axial or longitudinal extent of the body. One or more balloons 112, 114 (see e.g., FIG. 1) are included, which are configured to be inflated during a clinical situation, such as training or providing treatment to a subject. In some embodiments, the gastroesophageal tamponade 100 may have only a single esophageal balloon, or more than two balloons, to cite a few alternatives.

As used herein, the term "subject" is intended to include human and non-human animals, as well as test models or dummies used for training purposes. The terms "subject" and "patient" are used interchangeably and can refer to human patients, as well as non-human primates or experimental animals such as rabbits, dogs, cats, rats, mice, and other animals. Preferred subjects of the present disclosure include mammals, or more particularly, human patients in need of a treatment for a disease or disorder. A subject of the present disclosure may be a patient suffering from esophageal bleeding caused by varices.

In the embodiment illustrated in FIGS. 1-4, there is a gastric inflation port 116 having a gastric inflation lumen 118, a gastric aspiration port 120 having a gastric aspiration lumen 122, an esophageal inflation port 124 having an esophageal inflation lumen 126, and an esophageal aspiration port 128 having an esophageal aspiration lumen 130. In some embodiments, each port 116, 120, 124, 128 is an integral part of the body 102, and in other embodiments, it is feasible for each port to be a separate component that is then attached to or otherwise coupled with the axially extending body 102. Having the ports 116, 120, 124, 128 “coupled to” each lumen 118, 122, 126, 130 may include either embodiment, where there is attachment of a separate component, or an integral connection (e.g., all subcomponents are injection molded, 3-D printed, or otherwise formed as a unitary component). The ports 116, 120, 124, 128 are configured so as to radially extend from the body 102, and in this particular embodiment, they are equidistantly spaced around the outer perimeter 108. This spacing and arrangement can help with port differentiation during use of the gastroesophageal tamponade 100.

In some embodiments, colored ports 116, 120, 124, 128, that are advantageously labeled and correspond to an external legend, allow a user to differentiate the ports quickly in an intuitive, less-error prone fashion. Further, their arrangement does not get in the way of insertion, and they are able to resist malfunction as opposed to previously arranged and configured ports. The illustrated solution will give physicians two options for differentiating ports: they can look at the legend that may be included with the device 100 packaging, or they can read the labels on the ports 116, 120, 124, 128 themselves. In some embodiments, the labels will be larger and easier to read than the port labels that are currently used on tamponades. Labels may be engraved and colored black or white to be legible on each respective port 116, 120, 124, 128.

The color-coded and labeled ports 116, 120, 124, 128 can help the user distinguish the ports. One or more current tamponade designs have all red ports with small black labeling, making it difficult for the physician to differentiate, especially in the ER environment in which the labeling may be obscured by blood. The body 102 of the tamponade device 100 can be colored white so it can be differentiated from the ports 116, 120, 124, 128 and contrasted against the coloring of the oral cavity. The text on the ports 116, 120, 124, 128 can be black, bolded, and be surrounded by a white background.

In some embodiments, the ports 116, 120, 124, 128 will be color-coded to allow for quick differentiation. Utilizing color psychology, the gastric ports 116, 120 are designed to be red and yellow due to the ability for warm colors to stand out and provide a sense of urgency. With gastric inflation being the most important, this port 116 was chosen to be yellow because the color is the brightest. The gastric suction port 120 was chosen as red: even though the color is used in emergency situations such as exit signs and ambulance lights, there is a chance the red will blend in with the surrounding blood from the patient. Meanwhile, the esophageal ports 124, 128 were chosen to be cool colors (e.g., green and blue, respectively) to signify less significance and stand out less. The hue of green for the esophageal inflation port 124 that was chosen is very bright to indicate the importance of the esophageal balloon 114 if needed. However, to take color blindness into account, the gastric ports 116, 120 will be slightly larger, 100 mm as opposed to 70 mm long in one particular example, to indicate higher importance as a means of differentiating the ports other than color. The aspiration ports, both gastric 120 and esophageal 128, are slightly thicker (8 mm compared to 6 mm for the inflation ports 116, 124, for example) to use size again to identify between the port functions and accommodate Christmas tree connectors, which may be used to attach the ports to standard suction connectors in emergency rooms. This is contrary to at least some current tamponades, where the inflation ports are slightly longer than the aspiration ones. The inflation ports 116, 124 are configured in this embodiment to accommodate the stopcocks of a syringe, although other inflation mechanisms are certainly possible, such as a pressure canister or an automatic inflator, to cite a few examples.

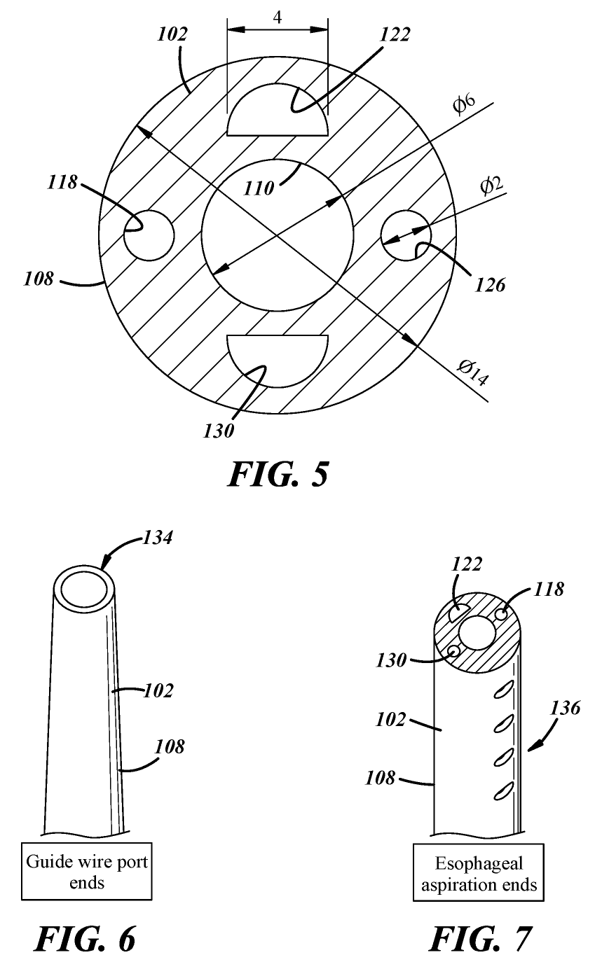

FIG. 5 shows an example cross-section view of the body 102 of the gastroesophageal tamponade 100. This view shows an advantageous arrangement and configuration for each of the lumens 110, 118, 122, 126, 130. In this arrangement, the guide lumen 110 is centrally located (i.e., a portion of the lumen extends through a middle 50% of the cross-sectional area within the outer perimeter 108). The guide lumen 110 is the largest of the lumens in this implementation, which as detailed below, will help with accommodating a guide wire 132 (not shown in FIG. 5). Incorporating the guide lumen 110 is technically challenging while maintaining the functionality of the other lumens within the size constraints of the device 100. Having the guide lumen 110 centrally located, particularly with a more flexible body 102 and a more rigid guide wire 132 can help improve deployment of the device 100. Further, in this embodiment, the guide lumen 110 and the inflation lumens 118, 126 are cylindrical shaped, whereas the aspiration lumens 122, 130 are hemispherical shaped. This can help increase the cross-sectional area of the aspiration lumens 122, 130 within the limited cross-sectional space. This is advantageous since the aspiration lumens 118, 126 accommodate more viscous fluid than the inflation lumens 118, 126 (e.g., blood vs. air). Additionally, the inflation lumens 118, 126 are symmetrical with respect to the guide lumen 110, and are diametrically opposed within the body 102. Similarly, the aspiration lumens 122, 130 are symmetrical with respect to the guide lumen 110, and are diametrically opposed within the body 102. This arrangement can help ultimately place each corresponding port 116, 120, 124, 128 in a location that is more intuitive for a user to differentiate, thereby making operation easier.

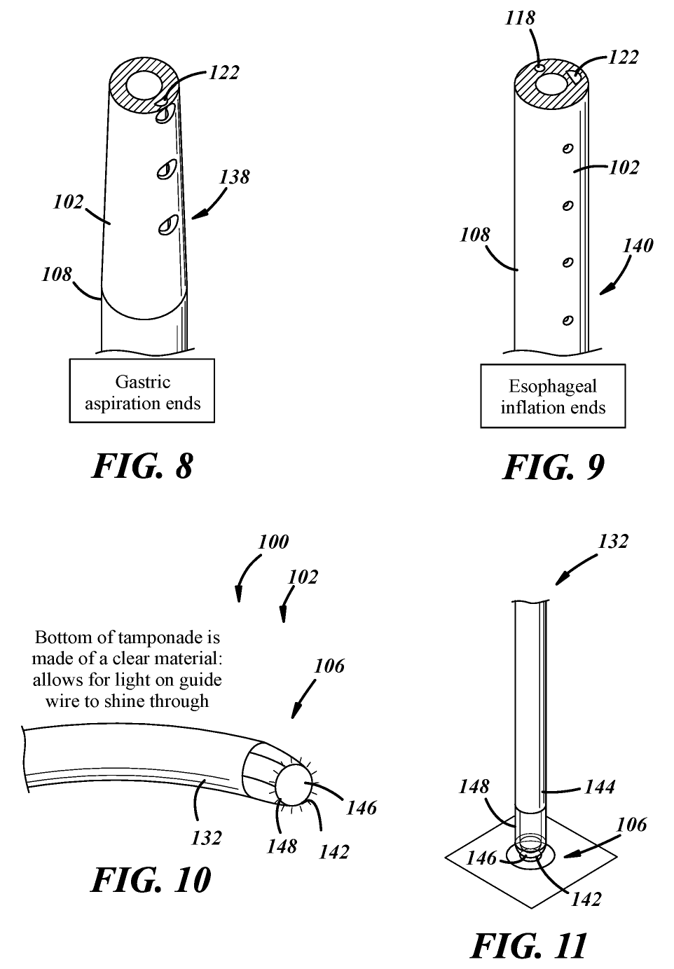

Each of the lumens 110, 118, 122, 126, 130 have one or more corresponding lumen ends 134, 136, 138, 140. FIG. 6 shows the guide lumen end 134 for the guide lumen 110, which is located at or toward the distal end 106 of the body 102. In this embodiment, the guide lumen 134 in this implementation spans the entire axial extent of the body 102 and ends at the distal end 106. This arrangement allows for the guide wire 132 to terminate at the distal end 106, or extend out from the distal end, which can enhance illumination when a light 142 is implemented on the guide wire 132 (see e.g., FIGS. 10 and 11 for features relating to the guide wire 132). In other embodiments, the guide lumen 110 may be closed at the distal end 102, and may include a transparent sheath, as detailed further below. FIG. 7 shows esophageal aspiration ends 136, which are located more toward the proximal end 104, and FIG. 8 shows gastric aspiration ends 138, which are located more toward the distal end 106. FIG. 9 shows example inflation ends 140, which may be configured similarly for the gastric inflation lumen 118 and the esophageal inflation lumen 136. However, the inflation ends for the gastric inflation lumen 118 are going to be located along the body 102 to inflate the gastric balloon 112, whereas the inflation ends 140 for the esophageal inflation lumen 126 are going to be located along the body to inflate the esophageal balloon 114 (FIG. 9 shows esophageal inflation ends 140, for example).

In one embodiment, the tamponade body 102 will be 14 mm in outer diameter with a 1 mm thickness at its thinnest point. This is slightly larger than currently available tamponades, which may be about 8 mm in luminal diameter and 12 mm total, to cite one example. The larger size of the tamponade 100 helps to accommodate the guide lumen 110 and guide wire 132, but as will be understood, the size constraints as dictated by the subject’s esophagus can make integration of the multi-function luminal structure difficult. The tamponade 100 is 95 cm in length along its axial extent. The distal end 106 is inserted into the patient’s mouth, while the proximal end 104 is the end that the physician will interact with. In one embodiment, the length of the esophageal balloon 114 is 22 cm, and the length of the gastric balloon 112 is 9 cm. Markers can be included along the length of the body 102 to help a user estimate a position of each of the balloons 112, 114.

In the illustrated embodiment of the tamponade 100, the cross-section of the aspiration lumens 122, 130 are hemispheres 3 mm in diameter. The cross-section of the inflation lumens 118, 126 are circles 1 mm in diameter. The lengths of these lumens from the ports 116, 120, 124, 128 at the proximal end 104 to the distal end 106 are as follows: 45 cm for esophageal aspiration 130, 66 cm for esophageal inflation 126, 76 cm for gastric inflation 118, and 84 cm for gastric aspiration 122. Each lumen 118, 122, 126, 130 terminates with additional holes at each of the ends 136, 138, 140 that connect the port hole 116, 120, 124, 128 to where the port is used; the esophageal suction lumen 130 terminates above (proximal to) the esophageal balloon 114, the esophageal inflation lumen 126 terminates within the esophageal balloon 114, the gastric inflation lumen 118 terminates within the gastric balloon 112, and the gastric aspiration lumen 122 terminates below (distal to) the gastric balloon 112. Other luminal end configurations are certainly possible.

The insertion end or distal end 106 of the device 100 should be configured to reach the stomach to allow for gastric balloon 112 anchoring. The port end or proximal end 104 of the device 100 should extend out of the mouth to allow physicians to inflate/deflate both the gastric and esophageal balloons 112, 114 as well as provide suction to the esophageal and gastric regions. The length from the incisor teeth to the esophagogastric junction generally ranges from 32-50 cm in adults. Thus, in order for the ports 116, 120, 124, 128 to be usable by the physician, the length of the body 102 should exceed 50 cm.

A somewhat flexible material can be used to make the body 102 and/or the balloons 112, 114. In one implementation, the flexible material is silicone, or more particularly, Shore 40A silicone. Other materials are certainly possible, such as SU-8, parylene, polyimide, and PDMS, to cite a few examples. The material for the body 102 is preferably rigid enough to help avoid coiling. In one embodiment, the material for the body 102 is more flexible than a more rigid material that is used for the guide wire 132. In one advantageous implementation, the material for the body 102 and the balloons 112, 114 is radiopaque. This may be accomplished during an injection molding manufacturing method for the body 102. During injection molding, the cast can include barium sulfate particulates, which will reflect x-rays and illustrate the exact position of not just the balloons 112, 114, but the entire body 102. This saves time and risk when clinicians with currently available tamponades have to partially inflate the balloon to identify it in an x-ray, which costs time and potentially injures the patient depending on the balloon's location at time of x-ray. Additionally, the outer perimeter 108 of the body 102 is configured to be smooth so that the tube can slide down the esophagus, as impaction caused by jagged edges or sharp corners could lacerate blood vessels, leading to hemorrhaging (e.g., a coefficient of kinetic friction less than 0.5 to avoid rupture stemming from abrasion from the insertion of the device 100).

FIGS. 10 and 11 illustrate embodiments that include a guide wire 132, and more particularly, a guide wire having a light source 142. The guide wire 132 can make it easier to insert the device into the patient, as it will help prevent coiling and give the physician more control over the distal end of the device. The shape, size, and configuration of the guide wire 132 may vary depending on the desired implementation, and will depend on the structure of the body 102 and guide lumen 110. In the illustrated embodiments, the guide wire 132 is similar in structure to a bougie: a thin, flexible surgical instrument to aid in dilating the trachea during intubation. An average bougie is 60 cm in length, 5 mm in diameter and curves at a 150° angle from the horizontal, which can be accommodated by the guide lumen 110 (e.g., about 6 mm in diameter). With the guide lumen 110 and guide wire 132 inside the body 102, a benefit to this is less friction to rub against the varices. However, this could be harder to fabricate because this would require taking into account the orientation of the guide lumen 110 in an already constricted space in relation to the other existing lumens, which are mere millimeters in diameter. Having the guide lumen 110 be centrally located can help accommodate the plurality of lumens 118, 122, 126, 130. In yet other embodiments, the guide lumen 110 may not be continuous or may be located at other locations with respect to the body 102 (e.g. portions of the guide wire 132 could be exposed along the axial extent of the body).

The guide wire 132 can provide stiffness to prevent coiling and bunching, but should be flexible enough that it does not overstrain or perforate the esophageal tissue. Such a wire 132 enables the tubing to be easily pushed down the esophagus with minimal risk of puncture. The guide wire 132 in the illustrated embodiment is a hollow cylinder 144 with a length of 100 cm, a luminal diameter of 5 mm with 0.5 mm thickness (making it 6 mm in diameter total). A white LED light 146 is used for the light source 142, and can be attached to the guide wire 132 at the distal end 106, with electrical wiring running through its hollow cylindrical cavity 144. The light will illuminate the oral cavity at the beginning of insertion to aid in identifying the esophagus, preventing the tamponade 100 from accidentally being inserted into the trachea. The white LED 146 in particular can be used to maximize the visibility inside the mouth, with a target lux of 1000 or greater while inside the mouth. Wiring to the LED light 146 can be safely contained within the guide wire 132, and it can be powered through a battery located toward proximal end 104 of the guide wire that remains outside of the body 102. Other configurations for the light source 142 are certainly possible, such as use of a waveguide, fiber optic, etc.

In one embodiment, a light source 142 is used that allows for the distinction between the esophagus and the trachea. The trachea is located on the anterior side of the body, while the esophagus is posterior; and the device 100 is inserted into the patient while they are on their back, so the trachea is facing the physician. This light 142 will quickly determine whether or not the distal end 106 of the device 100 has been inserted into the trachea and needs to be corrected, as the light will shine brightly through the trachea but not the esophagus. It will also provide visibility in the oral cavity. One goal of the light 142 is to make initial entrance into the esophagus simpler. The light 142 can also make navigation of the oral cavity easier and reduce the need for an external light source such as a laryngoscope. Furthermore, the light 142 can provide instant feedback to whether or not the device 100 has been inserted into the trachea instead of the esophagus. It may be feasible to implement a light 142 that confirms placement in the stomach, but the brightness should be enhanced while being mindful of temperature constraints, power needs, etc. (e.g., transdermal illumination may require 8000 lux or more).

A transparent or translucent sheath 148 can be included at the distal end 106 of the guide wire 132. This can help protect the light source 142 and enhance the transmission of light during use of the tamponade 100. In some implementations, particularly if the end of the guide lumen 110 is closed, there may also be a translucent or transparent sheath in the body 102 itself.

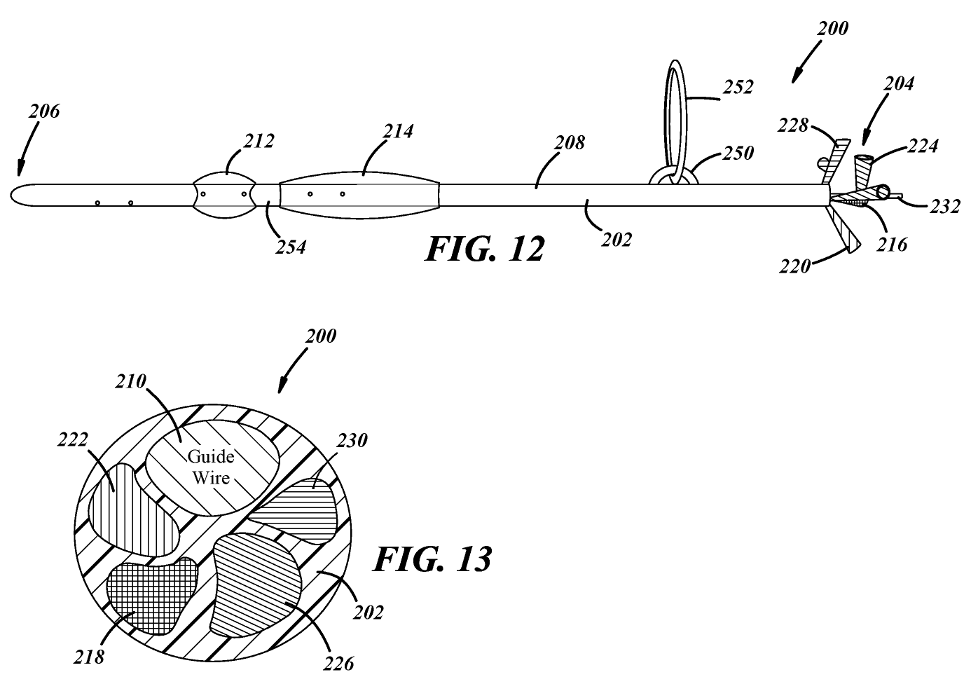

FIGS. 12-14 show other embodiments of gastroesophageal tamponades 200, 300 (like reference numerals denote like features, and teachings relating to one embodiment are applicable to other embodiments unless there is an incompatibility in features). With the tamponade 200 shown in FIGS. 12 and 13, the body 202 includes a hook 250 and a securing strap 252. In this embodiment, the external securing strap 252 is located approximately 10 cm away from the proximal end 204 at the ports 216, 220, 224, 228. The strap or loop 252 is about 40 mm in internal diameter, 12 mm in thickness, and 20 mm in height. The diameter of the strap 252 itself will be similar to that of a normal rubber band, which is 55 mm. However, the thickness will be larger, which will be 10 mm in one embodiment. The strap or loop 252 secures the proximal end 204 of the tamponade 200 to a prepackaged counterweight so that the weight of the inflated balloons 212, 214 does not pull the device 200 out of position in the patient’s esophageal tract. The counterweight (not shown) will be hooked onto the securing strap 252 of the tamponade 200 and can be hung over an IV pole to provide a constant 1-lb force, calibrated to not let the tamponade slip nor cause damage to the patient from the weight. Previous methods for external securement included placing a football helmet over the patient’s head, as well as tying a strip of gauze to the tamponade: these were proven to be inefficient, cumbersome, and overall served as a last resort for emergency physicians.

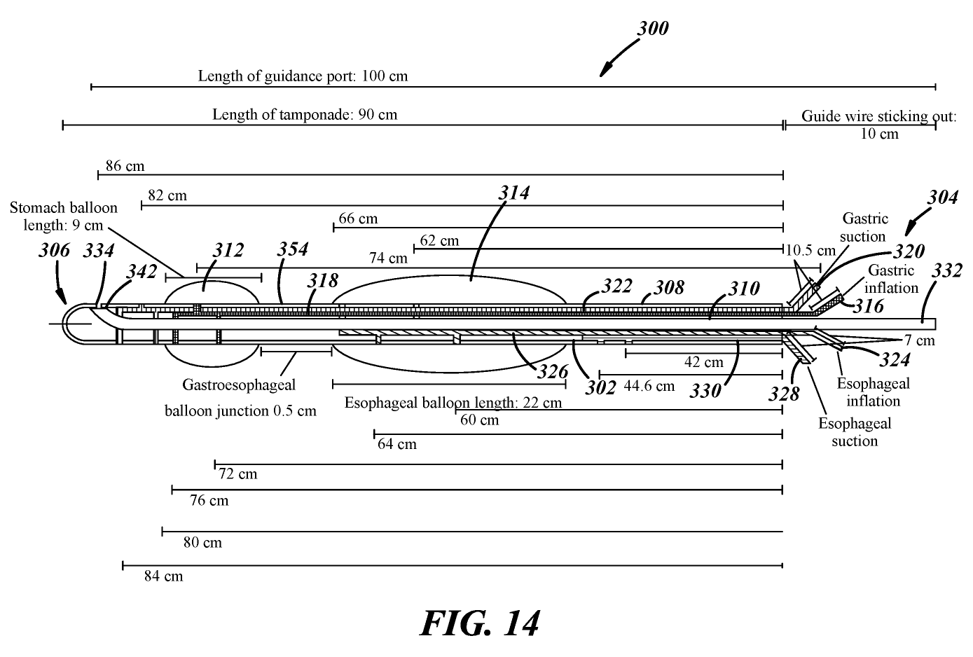

FIGS. 13 and 14 also show potential variations to the structure of the body 202, 302, and more particularly, changes to the lumen structure. With FIG. 13, the guide lumen 210 is located closer to the outer perimeter 208, but is still configured to allow adequate space for the other lumens 218, 222, 226, 230, which can be challenging given the overall size constraints of the device. In this implementation, the lumens 218, 222, 226, 230 have a more teardrop shape. In the embodiment of FIG. 14, the guide lumen 310 is a larger interstitial space within the body 310, and the other lumens are structured more as pipes within this interstitial space. Additionally, with the embodiment 300, the guide lumen 310 does not exit directly at the distal end 306, but instead, terminates along the side wall of the outer perimeter 308.

The embodiments 200, 300 also illustrate other structural features. For example, both implementations have a balloon junction space 254, 354, such that there is some separation along the axial extent of the body 202, 302 between the gastric balloon 212 and the esophageal balloon 214. In other embodiments, such as that shown in FIG. 1, there is no junction space, and even potentially some overlap between the balloons 112, 114. Additionally, as shown in more detail in FIG. 12, one or more of the ports 216, 220, 224, 228 may have a bifurcated structure, which may be used for a pressure gauge or the like.

In some implementations, the tamponade 100, 200, 300 is part of a kit with other supplies that may help with use and can serve to reduce prep time. Such supplies may include, but are not limited to, a syringe with a stopcock, medical Christmas trees, a counterweight, a pressurize cannister, a manometer, extra loops or straps for securing, a pH sensor, an external litmus strip, and an external legend. Two legends may be included, one attached to the inside of the box, and another one that is loose. In some embodiments, the tamponade 100, 200, 300 may be motorized or have some other deployment assistance mechanism.

Use of the tamponade will be discussed in relation to the tamponade 100, but the teachings are also applicable to the embodiments 200, 300 as well. The gastroesophageal tamponade 100 can be used to apply localized pressure to stop bleeding from gastrointestinal varices. When beginning insertion, the light source 142 allows for the distinction between the esophagus and the trachea. The trachea is located on the anterior side of the body, while the esophagus is posterior; the device 100 is inserted into the patient while they are on their back, so the trachea is facing the physician. This light will quickly determine whether or not the tip of the device has been inserted into the trachea and needs to be corrected, as the light will shine brightly through the trachea but not the esophagus.

The guide wire 132 can be implemented to make it easier to insert the tip of the device 100 into the stomach as well as provide illumination in the oral cavity with an LED 146 on the distal end 106. The balloons are inserted into the desired location (gastric balloon 112 and esophageal balloon 114 are placed in the stomach and esophagus, respectively) in the GI tract orally, and positioned over the bleeding site. The balloons 112, 114 can then be inflated manually through the use of syringes to apply direct pressure on the bleeding varix. The inflated balloons 112, 114 create a tamponade effect, compressing the blood vessels and preventing further bleeding. After the balloons 112, 114 are inflated, excess blood can be drained from the stomach and esophagus through gastric and esophageal suction ports 120, 128. The device 100 can then be secured using the external securing strap 152 and counterweight, pulling the device taught against the gastric balloon 112 in the stomach. The balloons 112, 114 may be left in place temporarily to allow time for the bleeding to stop and clot to form, before being deflated and removed when the patient has reached the operating room. The use of localized, controlled pressure from the inflated balloons 112, 114 to mechanically compress and occlude the bleeding source can provide time for hemostasis to occur.

The device 100 is intended to be used by emergency physicians in the emergency room. An example protocol for insertion is included below. The patient should be intubated and sedated before insertion of the device 100 if possible.

1.Retrieve procedure package from storage.

2.Deploy package contents onto the bedside table. Remove the port caps from the esophageal and gastric inflation ports 116, 124.

3.Call an x-ray technician so that they are on hand.

4.Hold the device 100 next to the patient, estimating where it would be located if inserted using anatomical knowledge. Take note of the external marking at the mouth.

5.Turn on the light 146 using the button on the battery pack attached to the top of the guide wire 132.

6.Insert the tip of the device 100 into the patient’s mouth.

7.Pass the tip of the device 100 through the oral cavity and into the esophagus, using the light 146 on the end of the device to ensure that it has not entered the trachea.

8.Keep inserting the device 100 into the esophagus until it has reached the designated mark on the tube at the mouth. The light 146 can be turned off once the device is in the esophagus.

9.Fill the gastric balloon 112 partially with 50 ccs of air using the yellow gastric inflation port 116 so that the balloon appears on an X-ray (this step may not be needed if a radiopaque material is used for the body 102 and/or balloons 112, 114).

10.Remove the guide wire 132 from the device 100.

11.Get an X-ray of the patient to ensure that the gastric balloon 112 has reached the stomach.

12.Attach the packaged syringe to the yellow gastric inflation port 116 (if not still attached from step 9) and inflate the gastric balloon 112 with 500 ccs of air. Remove the syringe and place the port cap back onto the port.

13.Attach the packaged Christmas tree to the red gastric suction port 120 and suction the stomach.

14.Attach the packaged Christmas tree to the blue esophageal suction port 128 and suction the esophagus. If bleeding is controlled (excess blood does not appear when applying suction), stop at this point.

15.If the patient is still bleeding, attach the syringe to the green esophageal inflation port 124 and inflate the esophageal balloon 114 to a pressure of up to 45 mmHg, confirmed via a manometer.

16.Remove the syringe and place the port cap on the esophageal inflation port 124.

17.Suction the esophagus again using the blue esophageal suction port 128.

18.Secure the external portion of the device 100 by attaching the counterweight to the securement strap 152 and looping it over an IV stand.

It is to be understood that the foregoing description is of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” "e.g.," “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all the following: “A”; “B”; “C”; “A and B”; “A and C”; “B and C”; and “A, B, and C.”

1. A gastroesophageal tamponade, comprising:
a body extending from a proximal end to a distal end, the body having an outer perimeter and a guide lumen extending at least partially through the body, the body comprising an aspiration lumen located at least partially between the guide lumen and the outer perimeter and an inflation lumen located at least partially between the guide lumen and the outer perimeter; and
a balloon extending at least partially around the body.

2. The gastroesophageal tamponade of claim 1, comprising a guide wire located at least partially within the guide lumen.

3. The gastroesophageal tamponade of claim 2, wherein the guide wire has a light source configured to illuminate an esophagus of a subject.

4. The gastroesophageal tamponade of claim 3, wherein the light source is at a distal end of the guide wire.

5. The gastroesophageal tamponade of claim 3, wherein the light source is a white light emitting diode (LED).

6. The gastroesophageal tamponade of claim 3, wherein the distal end of the body includes a transparent or translucent sheath.

7. The gastroesophageal tamponade of claim 1, wherein the inflation lumen is an esophageal inflation lumen and the balloon is an esophageal balloon.

8. The gastroesophageal tamponade of claim 7, further comprising a gastric inflation lumen and a gastric balloon.

9. The gastroesophageal tamponade of claim 8, wherein the gastric inflation lumen and the esophageal inflation lumen are diametrically opposed and the guide lumen is a centrally located guide lumen.

10. The gastroesophageal tamponade of claim 8, wherein the aspiration lumen is a gastric aspiration lumen and further comprising an esophageal aspiration lumen.

11. The gastroesophageal tamponade of claim 10, wherein the guide lumen is a centrally located guide lumen, and wherein the gastric inflation lumen and the esophageal inflation lumen are diametrically opposed, and the gastric aspiration lumen and the esophageal aspiration lumen are diametrically opposed.

12. The gastroesophageal tamponade of claim 10, wherein a gastric inflation port is coupled to the gastric inflation lumen, an esophageal inflation port is coupled to the esophageal inflation lumen, a gastric aspiration port is coupled to the gastric aspiration lumen, and an esophageal aspiration port is coupled to the esophageal aspiration lumen.

13. The gastroesophageal tamponade of claim 12, wherein the gastric inflation port and the gastric aspiration port are colored with one or more warm colors and the esophageal inflation port and the esophageal aspiration port are colored with one or more cool colors.

14. The gastroesophageal tamponade of claim 12, wherein the gastric inflation port, the esophageal inflation port, the gastric aspiration port, and the esophageal aspiration port are configured to extend radially outward from the body adjacent the guide lumen with the gastric inflation port being larger than the esophageal inflation port.

15. The gastroesophageal tamponade of claim 1, wherein the aspiration lumen has a cross-sectional shape and the inflation lumen has a cross-sectional shape, and wherein the cross-sectional shape of the aspiration lumen is different from the cross-sectional shape of the inflation lumen.

16. The gastroesophageal tamponade of claim 15, wherein a cross-sectional area of the cross-sectional shape of the aspiration lumen is larger than a cross-sectional area of the cross-sectional shape of the inflation lumen.

17. A gastroesophageal tamponade, comprising:
a body extending from a proximal end to a distal end, with a guide lumen extending at least partially through the body;
a balloon extending at least partially around the body;
a guide wire located at least partially within the guide lumen of the body; and
a light source configured to illuminate an esophagus of a patient.

18. The gastroesophageal tamponade of claim 17, wherein the guide lumen is a centrally located guide lumen and the body includes a plurality of aspiration lumens and a plurality of inflation lumens, wherein the plurality of aspiration lumens and the plurality of inflation lumens surround the centrally located guide lumen.

19. A method of using a gastroesophageal tamponade, the gastroesophageal tamponade comprising a body extending from a proximal end to a distal end and a light source configured to illuminate the distal end of the body, the method comprising the steps of:
illuminating a trachea and an esophagus of a subject with light from the light source; and
inserting the body of the gastroesophageal tamponade into the esophagus of the subject.
